(19) United States
(12) Patent Application Publication
(10) Pub. No.: US 2024/0350396 A1
(43) Pub. Date: Oct. 24, 2024

(54) SKIN PERFECTING AND TIGHTENING COMPOSITIONS

(71) Applicant: L'OREAL, Paris (FR)

(72) Inventors: Chunwei WU, Green Brook, NJ (US); Hy Si BUI, Piscataway, NJ (US)

(73) Assignee: L'OREAL, Paris (FR)

(21) Appl. No.: 18/637,919

(22) Filed: Apr. 17, 2024

Related U.S. Application Data
(60) Provisional application No. 63/496,799, filed on Apr. 18, 2023.

(30) Foreign Application Priority Data
Jun. 19, 2023 (FR) 2306254

(51) Int. Cl. A61K 8/73; A61K 8/06; A61K 8/25; A61Q 19/08

(52) U.S. Cl. CPC A61K 8/736; A61K 8/25; A61K 8/365; A61Q 19/08; A61K 2800/651; A61K 2800/95

(57) ABSTRACT

The instant disclosure is skin perfecting and tightening composition that provide an instantaneous and dramatic improvement to the appearance of skin, e.g., by reducing the appearance of wrinkles, eye bags, pores, and skin imperfections such as scarring, dark spots (and uneven skin tone), dark circles, and roughness. The skin perfecting and tightening compositions comprise: (a) one or more polysaccharides with amine groups; (b) silica particles; (c) one or more acids, salts thereof, or a combination thereof; (d) optionally, one or more fatty compounds; (e) optionally, one or more water-soluble solvents; and water. The skin perfecting and tightening compositions exhibit excellent transparency, haze, and gloss characteristics appropriate for application to the skin, which are particularly long lasting.

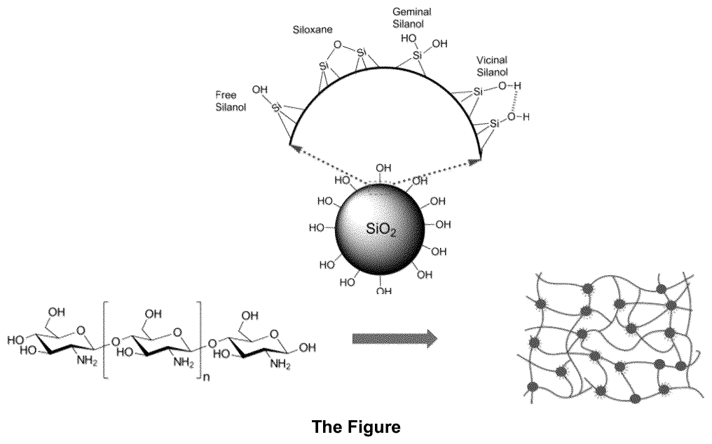

### CROSS-REFERENCE TO RELATED APPLICATIONS

This application claims benefit of U.S. Ser. No. 63/496,799, filed Apr. 18, 2023, and benefit of French Application No. FR 2306254, filed on Jun. 19, 2023, which are incorporated herein by reference in their entirety.

### FIELD OF THE DISCLOSURE

The present disclosure relates to skin perfecting and tightening compositions, and methods for improving the appearance of skin, for example, by providing a tightening, lifting, and smoothing effect to the skin.

### BACKGROUND

Skin produces less collagen and elastin as it ages. For example, after the age of twenty, a person (human) produces about 1 percent less collagen in the skin each year. As a result, the skin becomes thinner and more fragile. Inevitably, wrinkles, crow's feet, age-spots, eye bags, and the like, begin to form. In addition, many individuals suffer from scarring or other aberrations in the skin. Consumers often wish to improve the appearance of such age-related skin imperfections, preferably with instantaneous results. Many consumer products and procedures devoted to hiding and reducing wrinkles are available. Some products and procedures are simple and inexpensive, for example, applying make-up, particularly a primer or colored foundation, to cover the skin (and thereby cover and/or fill the wrinkles and provide a smoother look). Far more expensive and drastic procedures, such as surgical face lifts and Botox® injections, are also used to reduce the appearance of wrinkles. However, many consumers either cannot afford, or do not wish, to undergo such drastic cosmetic procedures. There are several lotions and creams that are formulated to hydrate the skin and make it more supple, thereby reducing the appearance of wrinkles. Some of these products contain active ingredients, for example, that help repair and rejuvenate skin over time. Unfortunately, many of these types of products suffer from various drawbacks.

Make-up products are often visible, offer minimal texture benefits, and have no long-lasting effect on the skin. After removal of the make-up, the skin looks the same as before the make-up was applied. Common skin care products can have chronic, acute or both effects on the skin. Hydration and optical effects are common acute benefits, but these benefits quickly wear-off over time.

Attempts have been made to develop new categories of products to improve the appearance of skin without the drawbacks of existing products and procedures. One such family of products can be generally classified as “adhesive, contractile film-forming polymers.” Film-forming polymers are chemical compositions that when applied to skin, leave a pliable, cohesive and continuous covering. A select group of film-forming polymers are also adhesive to the skin and contractile.

### SUMMARY OF THE DISCLOSURE

The instant disclosure relates to skin perfecting and tightening compositions for providing an instantaneous and dramatic improvement to the appearance of skin, e.g., by reducing the appearance of wrinkles, eye bags, pores, and skin imperfections such as scarring, dark spots (and uneven skin tone), dark circles, and roughness. Upon application to the skin, the compositions immediately begin tightening the skin while maintaining the skin's natural appearance, i.e., the treated skin does not appear to be manipulated or modified. The inventors discovered that the skin perfecting and tightening compositions exhibit excellent transparency, haze, and gloss characteristics appropriate for application to the skin, which are particularly long lasting. The skin perfecting and tightening compositions typically include:
(a) one or more polysaccharides with amine groups;
(b) silica particles, wherein (a) and (b) are in a weight ratio of about 10:1 to about 1:1;
(c) one or more acids, salts thereof, or a combination thereof;
(d) optionally, one or more fatty compounds, one or more water-soluble solvents, optionally, one or more thickening agents, one or more nonionic surfactants or emulsifiers; and
(e) water.

The one or more polysaccharides with amine groups include those with C₅-C₇ saccharide units and also the organic or mineral acid salts thereof, the a (alpha) or β (beta) anomers thereof, the optical isomers thereof of L or D configuration, and the solvates thereof such as hydrates. Nonlimiting examples, include polyhexosamines, polyglucosamines, or a combination thereof. In various embodiments, the one or more polysaccharides with amine groups comprises or consists of chitin, derivatives of chitin, chitosan, derivatives of chitosan, or a combination thereof.

Nonlimiting examples of silica particles include fumed silica, precipitated silica, or a combination thereof. Typically, the silica particles are hydrophilic and have not been surface treated with a hydrophobic component.

The one or more acids can be organic acids, inorganic acids, salts thereof, or a combination thereof. For example, useful acids include, but are not limited to those having one (mono), two (di), or three (tri) carboxylic acid groups (—COOH). The mono, di, and tricarboxylic acids, and/or salts thereof, typically have a molecular weight of less than about 500 g/mol, less than about 400 g/mol, or less than about 300 g/mol. In various preferred embodiments, the skin tightening and perfecting composition includes one or more alpha hydroxy acids. An alpha hydroxy acid is an organic acid containing a hydroxyl group bonded to the carbon atom adjacent to the carboxylic acid group. Nonlimiting examples include glycolic acid, lactic acid, tartaric acid, mandelic acid, malic acid, citric acid, phytic acid, hydroxycaprylic acid, hydroxycapric acid, a salt thereof, or a combination thereof, more preferably wherein the one or more alpha hydroxy acid comprises or consists of glycolic acid, a salt thereof, or a combination thereof.

Fatty compound can optionally be included in the skin perfecting and tightening compositions. Fatty compounds are lipophilic and may be derived, for example, from plant sources, animal sources, or petrochemical sources. In various embodiments, the one or more fatty compounds are preferably non-petrochemically derived. In further embodiments, the one or more fatty compounds are derived from plants or animals, preferably from plants. Nonlimiting examples of fatty compounds include oils, waxes, linear or branched alkanes, fatty esters, fatty alcohols, fatty acids, esters of fatty alcohols, esters of fatty acids, cetyl esters, triglycerides, or a mixture thereof.

The skin perfecting and tightening compositions optionally include one or more water soluble solvents (also referred to as “water-soluble organic solvents”). Nonlimiting examples include glycerin, C₁-C₆ mono-alcohols, polyols (polyhydric alcohols), glycols, or a mixture thereof. In various embodiments, at least one of the one or more water soluble solvents is glycerin, a glycol (e.g., ethylene glycol, propylene glycol, butylene glycol, pentylene glycol, caprylyl glycol, etc.) or a combination thereof.

The skin tightening and perfecting compositions may optionally include one or more thickening agents, depending on the desired thickness of the compositions.

Nonlimiting examples of thickening agents include carboxylic acid polymers, crosslinked polyacrylate polymers, polyacrylamide polymers, gums, and a combination thereof.

In various embodiments, the skin tightening and perfecting compositions may include one or more nonionic surfactants or emulsifiers. However, nonionic surfactants or emulsifiers are not required. Nonlimiting examples nonionic surfactants or emulsifiers include alkoxylated fatty alcohols, fatty acid esters of polyoxyethylene glycol, ethoxylated mono or diglycerides, sorbitan esters, ethoxylated sorbitan esters (polysorbates), fatty acid glycol esters, ethylene oxide, alkyl(ether)phosphates, alkylpolyglucosides, and mixtures thereof. In various embodiments, at least one of the one or more nonionic surfactants or emulsifiers is a polysorbates.

The skin perfecting and tightening compositions may optionally include one or more skin active agents. Nonlimiting examples (categories) of skin active agents include antioxidants, anti-wrinkle agents, depigmenting agents, vitamins, ceramides, alpha and/or beta hydroxy acids, anti-acne agents, etc.

The skin perfecting and tightening compositions may optionally include one or more miscellaneous ingredients. Nonlimiting examples include preservatives, fragrances, pH adjusters, salts, buffers, flavonoids, botanical extracts, UV filtering agents, proteins, protein hydrolysates and/or isolates, hydrotropes, pearlescent agents, fillers, colorants, mattifying agents, or a combination thereof.

The form of the skin perfecting and tightening composition is typically a liquid or liquid-like consistency, a serum, a gel or gel-like consistency, a lotion or a lotion-like consistency, a cream or a cream-like consistency, or a paste or a paste-like consistency. The form or consistency of the skin perfecting and tightening composition is such that it can be applied evenly over the skin. In various embodiments, the skin perfecting and tightening composition is in the form of an oil-in-water emulsion, a suspension, or a dispersion.

The instant disclosure also relates to methods for improving the appearance of skin comprising applying the compositions described herein to the skin. For example, the instant disclosure relates to methods for smoothing the skin comprising applying the compositions described herein to the skin and forming a film or layer on the skin. In various embodiments, the instant disclosure relates to methods for tightening the skin comprising applying the compositions described herein to the skin and forming a film or layer on the skin. In some instances, the compositions are applied to the skin of the face, and/or more specifically around the eyes, around the mouth, and/or around the neck of a human face. The methods of improving the appearance of skin include methods for treating or reducing the appearance of wrinkles, blemishes, dryness, roughness, dullness, age spots, scarring, divots, etc.

The compositions of the present disclosure are surprisingly stable, elastic, and provide an unexpectedly long-lasting skin smoothing and tightening effect. Unlike other products, the film or layer formed on the skin does not dry-out and whiten, crack, or peel. Instead, the film or layer remains flexible (elastic), durable, and comfortable. Moreover, the compositions (and resulting films or layers) hydrate and protect the underlying skin.

### BRIEF DESCRIPTION OF THE FIGURES

Implementation of the present technology will now be described, by way of example only, with reference to the attached figures, wherein:

THE FIGURE illustrates a mechanism by which it is believed the chitosan interacts with silica through intermolecular hydrogen bonding between surface silanol groups on the surface of the silica (—Si—OH) and side amine groups (—NH₂) and/or side hydroxyl groups (—OH) of chitosan.

It should be understood that the various aspects are not limited to the arrangements and instrumentality shown in the drawings.

### DETAILED DESCRIPTION OF THE DISCLOSURE

The present disclosure relates to skin perfecting and tightening compositions that provide instantaneous and long-lasting improvements to the appearance of skin. For example, the compositions provide an instantaneous smoothing and tightening effect to skin and are therefore useful for treating eye bags, facial wrinkles, and other age-related skin imperfections. Without wishing to be bound by any particular theory, it is believed the amine groups of the one or more polysaccharides with amine groups interact through intermolecular hydrogen bonding with silica surface silanol groups resulting in a film with a crosslinked polymeric network as the composition dries on a user's skin, thereby contributing to the films superb tensile and cohesive strength. The skin perfecting and tightening compositions are therefore particularly useful for filling and improving skin aberrations such as deep wrinkles, divots in the skin, scarring of the skin, etc. The skin perfecting and tightening compositions typically include:
(a) about 1 to about 12 wt. % of one or more polysaccharides with amine groups;
(b) about 0.1 to about 8 wt. % of silica particles, wherein (a) and (b) are in a weight ratio of about 6:1 to about 1:1;
(c) about 0.1 to about 5 wt. % of one or more acids, preferably one or more alpha hydroxy acids, salts thereof, or a combination thereof;
(d) optionally, about 0.1 to about 40 wt. % of one or more fatty compounds;
(e) optionally, about 0.01 to about 25 wt. % of one or more water-soluble solvents;
(f) optionally, about 0.01 to about 6 wt. % of one or more thickening agents;
(g) optionally, about 0.01 to about 5 wt. % of one or more nonionic surfactants or emulsifiers; and
(h) about 50 to about 90 wt. % of water;
   wherein all weight percentages are based on a total weight of the composition.
      (a) Polysaccharides with Amine Groups

The skin perfecting and tightening compositions include one or more polysaccharides with amine groups. The polysaccharides with amine group(s) may include organic or mineral acid salts thereof, the α or β anomers thereof, the optical isomers thereof of L or D configuration, and the solvates thereof such as hydrates. In some cases, the polysaccharide(s) with amine group(s) have a weight average molecular weight (MW) of less than or equal to 400 kDa, e.g. less than 200 kDa. Similarly, in some cases, the polysaccharide(s) with amine group(s) have a low weight average molecular weight, i.e. a MW<100 kDa, preferentially having a weight average MW which is <40 kDa, more preferentially having a weight average MW that is between 10 kDa and 200 kDa inclusive, more preferentially having a weight average MW that is between 20 kDa and 150 kDa inclusive.

In a preferred embodiment, the one or more polysaccharides with amine groups are linear or substantially linear. Furthermore, the one or more polysaccharides with amine groups are preferably soluble in water or substantially soluble in water at a temperature of 25° C. an atmospheric pressure. The phrase, “substantially soluble in water,” indicates that at least 90, 95, or 99 wt. % of the one or more polysaccharides with amine groups are solubilized in water at a temperature of 25° C. an atmospheric pressure.

The polysaccharide(s) with amine group(s) may be natural, of animal or plant origin, or derived from synthesis, hemisynthesis or biosynthesis. According to at least one particular embodiment, the polysaccharide(s) with amine group(s) are chosen from those with C₅-C₇ saccharide units and also the organic or mineral acid salts thereof, the α or β anomers thereof, the optical isomers thereof of L or D configuration, and the solvates thereof such as hydrates.

More particularly, the polysaccharide(s) with amine group(s) may be a C₆ saccharide unit with amine group(s). These polysaccharides with amine group(s) are generally referred to as polyhexosamines. According to at least one particular embodiment, the saccharide units of the polysaccharide with amine group(s) are of β (beta) anomeric configuration and/or D configuration.

In some cases, the saccharide units of the polysaccharide with amine group(s) are joined to one another between the C₁ carbon atoms of one saccharide unit and the C₄ carbon atoms of the other saccharide unit, denoted (1→4), such as the polysaccharide with amine group(s) of formula (B) below, and also the organic or mineral acid salts thereof, the α or β anomers thereof, the optical isomers thereof of L or D configuration, and the solvates thereof such as hydrates:

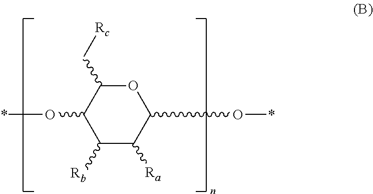

in which formula (B):
   the Rₐ, R_{b}, R_{c} radicals of each saccharide unit may be identical or different;
   n is an integer greater than or equal to 2, particularly between 3 and 3000 inclusive, and more particularly between 5 and 2500, preferentially between 10 and 2300;
   Rₐ, R_{b}, and R_{c}, which are identical or different, represent i) a hydroxyl group, ii) a (C₁-C₄)alkoxy group, the alkyl group of which may be optionally substituted, especially with one or more hydroxyl groups, iii) a carboxyl group, and iv) an NR₁R₂ group, with R₁ and R₂ as defined above, in particular R₁ and R₂ are chosen from a hydrogen atom and —C(O)—R′₁ in which R′₁ is as defined above; preferably R₁ and R₂ represent i) a hydrogen atom or ii) —C(O)—R′₁ with R′₁ representing a (C₁-C₄)alkyl group such as methyl;
   it being understood that at least one of the Rₐ, R_{b}, or R_{c} radicals of at least one saccharide unit represents an NR₁R₂ group and that at least one of the NR₁R₂ groups of at least one saccharide unit represents an NH₂ group; preferably Rₐ of at least one saccharide unit represents an NR₁R₂ group with R₁ which represents a hydrogen atom and R₂ is chosen from i) a hydrogen atom or ii) a —C(O)—R′₁ group, and R_{b} and R_{c} represent a hydroxyl group, it being understood that at least one of the NR₁R₂ groups of at least one saccharide unit represents an NH₂ group.

More particularly, the polysaccharide(s) with amine group(s) are of formula (B₁) below, and also the organic or mineral acid salts thereof, the α or β anomers thereof, the optical isomers thereof of L or D configuration, and the solvates thereof such as hydrates:

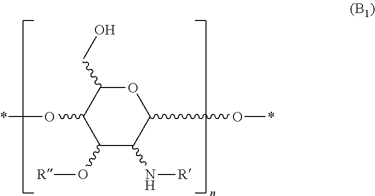

in which formula (B1):
   R′ represents a hydrogen atom or a (C₁-C₄)alkylcarbonyl group such as acetyl CH₃—C(O)—;
   R″ represents a hydrogen atom or a (C₁-C₄)alkyl group optionally substituted with a carboxyl group such as —CH(CO₂H)—CH₃;
   n is an integer greater than or equal to 2, particularly between 3 and 3000 inclusive, more particularly between 5 and 2500, preferentially between 10 and 2300; it being understood that in the polysaccharide (B₁) at least one saccharide unit bears an NH₂ amino group and at least one other saccharide unit bears at least one N(H)—R′ group with R′ representing a (C₁-C₄)alkylcarbonyl group such as acetyl CH₃—C(O)—.

Preferably, the saccharide units of formula (B) or (B₁) are of D configuration, also referred to as D-glucopyran. The units of formula (B) or (B₁) are particularly of R (beta) anomeric configuration. According to one particular embodiment, the polysaccharides of the invention are chosen from the compounds of formula (B₂) below and also the organic or mineral acid salts thereof, and the solvates thereof such as hydrates:

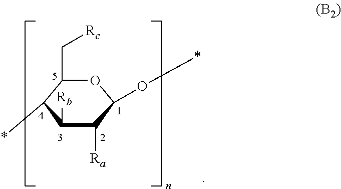

in which formula (B₂):
   Rₐ, R_{b}, and R_{c}, are as defined for (B) above; and
   the Rₐ, R_{b}, R_{c} radicals of each saccharide unit may be identical or different;
   n is an integer greater than or equal to 2, particularly between 3 and 3000 inclusive, more particularly between 5 and 2500, preferentially between 10 and 2300; it being understood that in the polysaccharide (B₂) at least one of the Rₐ, R_{b}, or R_{c} radicals of at least one saccharide unit represents an NR₁R₂ group and that at least one of the NR₁R₂ groups of at least one saccharide unit represents an NH₂ group; preferably at least one saccharide unit bears an Rₐ amino NH₂ group and at least one other saccharide unit bears an Rₐ group which represents —N(H)—R′ with R′ representing a (C₁-C₄)alkylcarbonyl group such as acetyl CH₃—C(O)—.

Preferentially, the polysaccharide(s) with amine group(s) are chosen from chitin and chitosan and their derivatives, preferably chitosan. Chitosan can be vegetable sourced (commercially known by the tradename of KIONUTRIME CSG, supplied by the company Kitozyme), or derived, for example, by treating the chitin shells of shrimp and other crustaceans with an alkaline substance, like sodium hydroxide.

More particularly, the polysaccharide(s) with amine group(s) are chosen from those of formula (B₃) below, and also the organic or mineral acid salts thereof, and the solvates thereof such as hydrates:

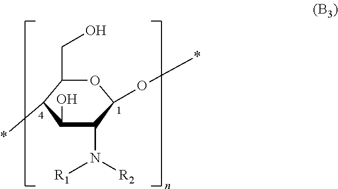

in which formula (B₃):
   R₁ and R₂ are as defined in the formula (B), (B1) or (B2); and
   n is an integer greater than or equal to 2, particularly between 3 and 3000 inclusive, more particularly between 5 and 2500, preferentially between 10 and 2300; it being understood that in the polysaccharide of formula (B₃) at least one saccharide unit bears an NH₂ amino group and at least one other saccharide unit bears an N(H)—R′ group with R′ representing a (C₁-C₄)alkylcarbonyl group such as acetyl CH₃—C(O)—.

More particularly, the polysaccharide(s) with amine group(s) are chosen from chitosans of formula (B₄) below, and also the organic or mineral acid salts thereof, and the solvates thereof such as hydrates:

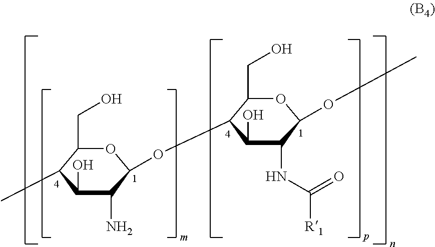

in which formula (B₄):
   R′₁ representing a (C₁-C₄)alkyl group such as methyl; and
   n is an integer greater than or equal to 2, particularly between 3 and 3000 inclusive, more particularly between 5 and 2500, preferentially between 10 and 2300;
   P is greater than 0 and ranges up to 0.5, preferably from 0.05 to 0.3, and better still from 0.1 to 0.20 such as 0.15 with m+p being equal to 1; it being understood that in the chitosan at least one saccharide unit bears an NH₂ amino group and at least one other saccharide unit bears an N(H)—R′₁ group with R′ representing a (C₁-C₄)alkylcarbonyl group such as acetyl CH₃—C(O)—.

For example, when m=0.7, p=0.3 this means that 70% of the amine groups are free (unsubstituted) and 30% of the amino groups are N-alkyl(C₁-C₄)carbonyl groups, in particular N-acetyl groups, corresponding to the chitosan of formula:

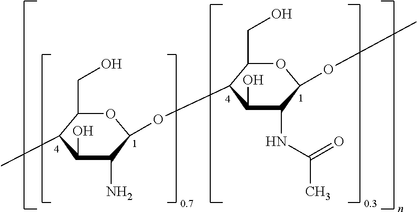

with n as defined above.

In some instances, the polysaccharide(s) with amine group(s) are chosen from chitosans, salified using organic acid, preferentially using monocarboxylic acid of formula (I) as defined above or polycarboxylic acid of formula (II) as defined above, more preferentially salified using carboxylic acid of formula (I) such as lactic acid.

In some cases, the polysaccharide(s) with amine group(s) refers to a mixture of polysaccharide(s) with amine group(s), one of which is a chitosan or the organic or mineral acid salts thereof, preferably the salts thereof of an organic acid such as lactic acid, the α or β anomers thereof, the optical isomers thereof of L or D configuration, and the solvates thereof such as hydrates. Alternatively, the polysaccharide(s) with amine group(s) may relate to a single polysaccharide with amine group(s), in particular a mixture of chitosan or the organic or mineral salts thereof or more particularly the organic acid salts thereof such as the lactic acid salt thereof, the α or β anomers thereof, the optical isomers thereof of L or D configuration, and the solvates thereof such as hydrates.

According to at least one embodiment, the polysaccharide(s) with amine group(s) denotes a single polysaccharide with amine group(s), in particular a chitosan or the organic or mineral acid salts thereof or more particularly the organic acid salts thereof such as the lactic acid salt thereof, the α or β anomers thereof, the optical isomers thereof of L or D configuration, and the solvates thereof such as hydrates.

In a preferred embodiment at the one or more polysaccharides with amine groups comprises or consists of chitosan is a β1,4-linked glucosamine polymer. Chitosan is the commonly used name for poly-[1-4]-β-D-glucosamine. Chitosan is chemically derived from chitin, which is a poly-[1-4]-β-N-acetyl-D-glucosamine, and which, in turn, is derived from the cell walls of fungi, the shells of insects and, especially, crustaceans. Chitin is treated with strong alkalis to remove acetyl groups producing chitosan. Depending on the specific treatment of chitin, chitosan can vary in the degree of deacetylation. Subsequent treatment with mineral acids or enzymes is used to break down the natural chitosan polymers (up to around 1.2 megaDaltons in size) into shorter β1,4-linked 2-amino-2-deoxy-glucopyranosyl polymers. Chitosans obtained in this manner generally contain polymers that include a range of sizes, including polymers that are larger than 10,000 Daltons in size and are insoluble in aqueous media, but can be dissolved by converting the glucosamine residue to its acid salt.

As already mentioned, chitosan can be obtained from N-deacetylation of chitin. Both these polysaccharides are copolymers of β, (1→4) linked N-acetyl-d-glucosamine and d-glucosamine units. The degree of acetylation represents the proportion of N-acetyl-d-glucosamine units with respect to the total number of units. It allows for distinction between chitin and chitosan. In the case of chitosan, degree of acetylation is can be below 50%. This value also influences the solubility limit of the polymer in dilute acidic solutions (2<pH<6). For purposes of the present invention, “chitosan,” means any copolymer formed of constituent units N-acetyl-D-glucosamine and D-glucosamine, whose degree of acetylation is less than 90%, preferably less than 80%, preferably less than 70%, preferably less than 60%, preferably less than 50%. In further embodiments, the degree of acetylation is sufficient to ensure solubility of at least 70%, preferably at least 80%, preferably at least 90%, preferably at least 95%, preferably at least 99% of the chitosan in the skin tightening and perfecting composition at 25° C.

In various embodiments, the chitosan has a weight average molecular weight (MW) greater than or equal to 10 kDa or greater than or equal to 15 kDa. In further embodiments, the chitosan has a weight average molecular molecular weight of about kDa to about 1 MDa, about 10 kDa to about 500 kDa, about 10 kDa to about 250 kDa, about 10 kDa to about 100 kDa, about 15 kDa to about 1 MDa, about 15 kDa to about 500 kDa, about 15 kDa to about 250 kDa, about 15 kDa to about 100 kDa.

In a preferred embodiment, the chitosan is linear or substantially linear. Furthermore, the chitosan is preferably soluble in water or substantially soluble in water at a temperature of 25° C. an atmospheric pressure. The phrase, “substantially soluble in water,” indicates that at least 90, 95, or 99 wt. % of the chitosan is solubilized in water at a temperature of 25° C. an atmospheric pressure.

The total amount of the one or more polysaccharides with amine groups will vary. Nonetheless, in various embodiments, the skin perfecting and tightening composition includes about 1 to about 12 wt. %, based on a total weight of the composition. In further embodiments, the skin perfecting and tightening composition includes about 1 to about 10 wt. %, about 1 to about 8 wt. %, about 1 to about 6 wt. %, about 2 to about 12 wt. %, about 2 to about 10 wt. %, about 2 to about 8 wt. %, about 2 to about 6 wt. %, about 3 to about 12 wt. %, about 3 to about 10 wt. %, about 3 to about 8 wt. %, or about 3 to about 6 wt. %, based on a total weight of the composition.

### (b) Silica Particles

The skin perfecting and tightening composition includes silica particles. In preferred embodiments, the silica particles are hydrophilic, e.g., they have not been hydrophobically modified. Nonlimiting examples of silicas fumed silica, precipitated silica, or a combination thereof. Preferably, the silica particles nanoparticles or microparticles. In various embodiments, the skin perfecting and tightening compositions include hydrophilic silica particles, wherein the hydrophilic silica particles have not been surface treated to alter surface properties of the silica particles. In various embodiments, the silica particles have an average diameter of about 1 μm to about 50 μm. If further embodiments, the silica particles have an average diameter of about 1 μm to about 30 μm, about 1 μm to about 20 μm, about 1 μm to about 5 μm, about 2 μm to about 50 μm, about 2 μm to about 40 μm, about 2 μm to about 30 μm, or about 2 μm to about 20 μm. Notwithstanding the diameters referenced above, silica particles that can be dispersed in water without aggregation are useful regardless of particles size. Both nano particles and micro particles can be used.

The total amount of the silica particles will vary. Nonetheless, in various embodiments, the skin perfecting and tightening compositions include about 0.5 to about 8 wt. % of silica particles, based on a total weight of the composition. In further embodiments, the skin perfecting and tightening composition includes about 0.5 to about 6 wt. %, about 0.5 to about 5 wt. %, about 0.5 to about 4 wt. %, about 0.5 to about 3 wt. %, about 1 to about 8 wt. %, about 1 to about 6 wt. %, about 1 to about 5 wt. %, about 1 to about 4 wt. %, about 1 to about 3 wt. %, about 1.5 to about 8 wt. %, about 1.5 to about 6 wt. %, about 1.5 to about 5 wt. %, about 1.5 to about 4 wt. %, or about 1.5 to about 3 wt. % of silica particles, based on a total weight of the composition.

### (c) Acids

The one or more acids can be organic acids, inorganic acids, or a combination thereof. The one or more acids may include mineral and/or organic acids, such as hydrochloric acid, orthophosphoric acid, sulfuric acid, sulfonic acids, and carboxylic acids. Examples of carboxylic acids, include, for instance, acetic acid, tartaric acid, citric acid, and lactic acid. In various embodiments, acids have one (mono), two (di), or three (tri) carboxylic acid groups (—COOH). The mono, di, and tricarboxylic acids, and/or salts thereof, typically have a molecular weight of less than about 500 g/mol, less than about 400 g/mol, or less than about 300 g/mol

Non-limiting examples of monocarboxylic acids include formic acid, acetic acid, propionic acid, butyric acid, valeric acid, caproic acid, entanthic acid, caprylic acid, pelargonic acid, capric acid, undecylic acid, lauric acid, tridecylic acid, lauric acid, tridecylic acid, myristic acid, pentadecylic acid, palmitic acid, margaric acid, stearic acid, nonadecylic acid, arachidic acid, lactic acid, a salt thereof, and a mixture thereof.

Non-limiting examples of dicarboxylic acids include oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, a salt thereof, and a mixture thereof.

Non-limiting examples of tricarboxylic acids include citric acid, isocitric acid, aconitric acid, propane-1,2,3-tricarboxylic acid, benzene-1,3,5-tricarboxylic acid, a salt thereof, and a mixture thereof.

The total amount of the one or more acids in the skin perfecting and tightening compositions will vary. Nonetheless, in various embodiments, the skin perfecting and tightening composition includes about 0.1 to about 8 wt. % of the one or more acids, salts thereof, or mixtures thereof. In further embodiments, the skin perfecting and tightening composition includes about 0.1 to about 6 wt. %, about 0.1 to about 5 wt. %, about 0.1 to about 4 wt. %, about 0.1 to about 3 wt. %, about 0.5 to about 8 wt. %, about 0.5 to about 6 wt. %, about 0.5 to about 5 wt. %, about 0.5 to about 4 wt. %, about 0.5 to about 3 wt. %, about 1 to about 8 wt. %, about 1 to about 6 wt. %, about 1 to about 5 wt. %, about 1 to about 4 wt. %, about 1 to about 3 wt. %, or about 1 to about 2 wt. % of the one or more acids, based on a total weight of the composition.

In various preferred embodiments, the skin tightening and perfecting composition includes one or more alpha hydroxy acids. An alpha hydroxy acid is an organic acid containing a hydroxyl group bonded to the carbon atom adjacent to the carboxylic acid group. The skin perfecting and tightening compositions of the instant disclosure may include one or more alpha hydroxy acids, salts thereof, or a combination thereof. Nonlimiting examples of alpha hydroxy acids (a-hydroxy acids or AHA) include lactic acid, citric acid, methyllactic acid, glucuronic acid, glycolic acid, pyruvic acid, 2-hydroxybutanoic acid, 2-hydroxypentanoic acid, 2-hydroxyhexanoic acid, 2-hydroxyheptanoic acid, 2-hydroxyoctanoic acid, 2-hydroxy-nonanoic acid, 2-hydroxydecanoic acid, 2-hydroxyundecanoic acid, 2-hydroxydodecanoic acid, acid 2-hydroxytetradecanoic, 2-hydroxy-hexadecanoic acid, 2-hydroxyoctadecanoic acid, 2-hydroxytetra-cosanoic acid, 2-hydroxyeicosanoic acid, mandelic acid, phenyllactic acid, gluconic acid, galacturonic acid, aleuritic acid, ribonic acid, tartronic acid, tartaric acid, malic acid, fumaric acid, salts thereof, or mixtures thereof. In a preferred embodiments, the one or more alpha hydroxy acids are selected from glycolic acid, lactic acid, tartaric acid, mandelic acid, malic acid, citric acid, phytic acid, hydroxycaprylic acid, hydroxycapric acid, a salt thereof, or a combination thereof. In a preferred embodiment, at least one of the one or more alpha hydroxy acids, salts thereof, or a combination thereof is glycolic acid, a salt thereof, or a combination thereof.

The total amount of the one or more alpha hydroxy acids in the skin perfecting and tightening compositions will vary. Nonetheless, in various embodiments, the skin perfecting and tightening composition includes about 0.1 to about 8 wt. % of the one or more alpha hydroxy acids, salts thereof, or mixtures thereof. In further embodiments, the skin perfecting and tightening composition includes about 0.1 to about 6 wt. %, about 0.1 to about 5 wt. %, about 0.1 to about 4 wt. %, about 0.1 to about 3 wt. %, about 0.5 to about 8 wt. %, about 0.5 to about 6 wt. %, about 0.5 to about 5 wt. %, about 0.5 to about 4 wt. %, about 0.5 to about 3 wt. %, about 1 to about 8 wt. %, about 1 to about 6 wt. %, about 1 to about 5 wt. %, about 1 to about 4 wt. %, about 1 to about 3 wt. %, or about 1 to about 2 wt. %, based on a total weight of the composition.

### (d) Fatty Compounds

The term “fatty compound” means an organic compound that is insoluble in water at ordinary temperature (25° C.) and at atmospheric pressure (760 mmHg), i.e. which has a solubility of less than 5%, preferably less than 1% and even more preferentially less than 0.1%. They have in their structure a hydrocarbon-based chain containing at least 6 carbon atoms.

In various embodiments, the one or more fatty compounds are preferably non-petrochemically derived. In further embodiments, the one or more fatty compounds are derived from plants or animals, preferably from plants.

The total amount of the one or more fatty compounds in the leave-on compositions, if present, will vary. Nonetheless, in various embodiments, the skin perfecting and tightening compositions include about 1 to about 40 wt. % of the one or more fatty compounds, based on the total weight of the compositions. In further embodiments, the skin perfecting and tightening compositions include about 1 to about 30 wt. %, about 1 to about 20 wt. %, about 1 to about 15 wt. %, about 1 to about 10 wt. %, about 1 to about 8 wt. %, about 2 to about 40 wt. %, about 2 to about 30 wt. %, about 2 to about 20 wt. %, about 2 to about 15 wt. %, about 2 to about 10 wt. %, about 2 to about 8 wt. %, about 3 to about 40 wt. %, about 3 to about 30 wt. %, about 3 to about 20 wt. %, about 3 to about 15 wt. %, about 3 to about 10 wt. %, or about 3 to about 8 wt. %, based on a total weight of the composition.

### (e) Water-Soluble Organic Solvents

The term “water soluble organic solvent” is interchangeable with the terms “water soluble solvent” and “water-miscible solvent” and means a compound that is liquid at 25° C. and at atmospheric pressure (760 mmHg), and it has a solubility of at least 50% in water under these conditions. In some cases, the water-soluble solvents has a solubility of at least 60%, 70%, 80%, or 90%. Non-limiting examples of water-soluble solvents include, for example, organic solvents selected from glycerin, alcohols (for example C₁₋₈, or C₁₋₄ alcohols), polyols (polyhydric alcohols), glycols, and a mixture thereof.

Nonlimiting examples of water-soluble organic solvents. Non-limiting examples of water-soluble organic solvents include, for example, organic solvents selected from glycerin, alcohols (for example, C₁₋₁₀, C₁₋₈, or C₁₋₄ alcohols), polyols (polyhydric alcohols), glycols, and a mixture thereof. Nonlimiting examples of monoalcohols and polyols include ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin.

Further non-limiting examples of water soluble organic solvents include alkanediols (polyhydric alcohols) such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

Polyhydric alcohols are useful. Examples of polyhydric alcohols include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a mixture thereof.

In a preferred embodiment, the skin perfecting and tightening compositions include glycerin, ethylene glycol, propylene glycol, butylene glycol, pentylene glycol, hexylene glycol, caprylyl glycol, dipropylene glycol, or a mixture thereof.

The total amount of the one or more water soluble solvents in the compositions, if present, will vary. Nonetheless, in various embodiments, the compositions include about 0.1 to about 25 wt. % of the one or more water soluble solvents, based on the total weight of the compositions. In further embodiments, the compositions include about 0.1 to about 20 wt. %, about 0.1 to about 15 wt. %, about 0.1 to about 10 wt. %, about 0.1 to about 8 wt. %, about 0.1 to about 5 wt. %, about 0.5 to about 25 wt. %, about 0.5 to about 20 wt. %, about 0.5 to about 15 wt. %, about 0.5 to about 10 wt. %, about 0.5 to about 8 wt. %, or about 0.5 to about 5 wt. %, based on the total weight of the compositions.

### (f) Thickening Agents

The total amount of the one or more thickening agents will vary. Nonetheless, in various embodiments, the skin tightening and perfecting composition includes about 0.1 to about 8 wt. % of the one or more thickening agents, based on the total weight of the composition. In further embodiments, the leave-on compositions include about 0.1 to about 6 wt. %, about 0.1 to about 5 wt. %, about 0.1 to about 4 wt. %, about 0.1 to about 3 wt. %, about 0.1 to about 2 wt. %, about 0.2 to about 8 wt. %, about 0.2 to about 6 wt. %, about 0.2 to about 5 wt. %, about 0.2 to about 4 wt. %, about 0.2 to about 3 wt. %, about 0.2 to about 2 wt. %, about 0.3 to about 8 wt. %, about 0.3 to about 6 wt. %, about 0.3 to about 5 wt. %, about 0.3 to about 5 wt. %, about 0.3 to about 4 wt. %, about 0.3 to about 3 wt. %, about 0.3 to about 2 wt. %, about 0.5 to about 8 wt. %, about 0.5 to about 5 wt. %, about 0.5 to about 4 wt. %, about 0.5 to about 3 wt. %, about 0.5 to about 2 wt. %, about 1 to about 8 wt. %, about 1 to about 5 wt. %, about 1 to about 4 wt. %, about 1 to about 3 wt. %, or about 1 to about 2 wt. % of the one or more thickening agents, based on a total weight of the composition.

### (g) Nonionic Surfactant or Emulsifier

The terms “nonionic surfactant” and “nonionic emulsifier” are used interchangeably in the instant disclosure and therefore can be referred to as “nonionic emulsifying surfactants.” The nonionic surfactant or emulsifier may have an HLB (hydrophilic-lipophilic balance) ranging from 1 to 7.9 or greater than or equal to 8. “HLB” refers to the “hydrophilic-lipophilic balance” associated with nonionic surfactants or emulsifiers. In particular, “HLB” value relates to the ratio of hydrophilic groups and lipophilic groups in emulsifiers, and also relates to solubility of the emulsifiers. Lower HLB emulsifiers (such as those with HLB values ranging from 1 to 7.9) are more soluble in oils (lipophilic material) and are more appropriate for use in water-in-oil (W/O) emulsions. Higher HLB emulsifiers (such as those with HLB values higher than 8) are more soluble in water (hydrophilic material) and are more appropriate for oil-in-water (O/W) emulsions.

Nonlimiting examples of nonionic surfactants or emulsifiers include alkyl and polyalkyl esters of poly(ethylene oxide), alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, alkyl and polyalkyl glycosides or polyglycosides, in particular alkyl and polyalkyl glucosides or polyglucosides, alkyl and polyalkyl esters of sucrose, optionally polyoxyethylenated alkyl and polyalkyl esters of glycerol, and optionally polyoxyethylenated alkyl and polyalkyl ethers of glycerol, and mixtures thereof. Preferably, the non-ionic surfactant(s) may be chosen from alkyl and polyalkyl esters of poly(ethylene oxide), alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, optionally polyoxyethylenated alkyl and polyalkyl esters of glycerol, and optionally polyoxyethylenated alkyl and polyalkyl ethers of glycerol, and mixtures thereof.
(1) Alkyl and polyalkyl esters of poly(ethylene oxide) that are preferably used are those containing at least one C₈-C₃₀ alkyl radical, with a number of ethylene oxide (EO) units ranging from 2 to 200. Mention may be made, for example, of (INCI name) PEG-20 stearate, PEG-40 stearate, PEG-100 stearate, PEG-20 laurate, PEG-8 laurate, PEG-40 laurate, PEG-150 distearate, PEG-7 cocoate, PEG-9 cococate, PEG-8 oleate, PEG-10 oleate and PEG-40 hydrogenated castor oil.
(2) Alkyl and polyalkyl ethers of poly(ethylene oxide) that are preferably used are those containing at least one C₈-C₃₀ alkyl radical, with a number of ethylene oxide (EO) units ranging from 3 to 200. Mention may be made, for example, of laureth-3, laureth-4, laureth-7, laureth-23, ceteth-5, ceteth-7, ceteth-15, ceteth-23, oleth-5, oleth-7, oleth-10, oleth-12, oleth-20, oleth-50, phytosterol 30 EO, steareth-6, steareth-20, steareth-21, steareth-40, steareth-100, beheneth 100, ceteareth-7, ceteareth-10, ceteareth-15, ceteareth-25, pareth-3, pareth-23, C₁₂-15 pareth-3, C₁₂-13 pareth-4, C₁₂-13 pareth-23, trideceth-3, trideceth-4, trideceth-5, trideceth-6, trideceth-7 and trideceth-10, and mixtures thereof.
(3) Polyoxyethylenated alkyl and polyalkyl esters of sorbitan that are preferably used are those with a number of ethylene oxide (EO) units ranging from 0 to Mention may be made, for example, of sorbitan laurate, sorbitan laurate 4 EO, sorbitan laurate 20 EO (polysorbate 20), sorbitan palmitate 20 EO (polysorbate 40), sorbitan stearate 20 EO (polysorbate 60), sorbitan oleate 20 EO (polysorbate 80) and sorbitan trioleate 20 EO (polysorbate 85).
(4) Polyoxyethylenated alkyl and polyalkyl ethers of sorbitan that are preferably used are those with a number of ethylene oxide (EO) units ranging from 0 to

The compositions of the instant disclosure may include one or more alkanolamides. Non-limiting examples alkanolamides include fatty acid alkanolamides. The fatty acid alkanolamides may be fatty acid monoalkanolamides or fatty acid dialkanolamides or fatty acid isoalkanolamides, and may have a C₂-8 hydroxyalkyl group (the C₂-8 chain can be substituted with one or more than one —OH group). Non-limiting examples include fatty acid diethanolamides (DEA) or fatty acid monoethanolamides (MEA), fatty acid monoisopropanolamides (MIPA), fatty acid diisopropanolamides (DIPA), and fatty acid glucamides (acyl glucamides).

Suitable fatty acid alkanolamides include those formed by reacting an alkanolamine and a C₆-C₃₆ fatty acid. Examples include, but are not limited to: oleic acid diethanolamide, myristic acid monoethanolamide, soya fatty acids diethanolamide, stearic acid ethanolamide, oleic acid monoisopropanolamide, linoleic acid diethanolamide, stearic acid monoethanolamide (Stearamide MEA), behenic acid monoethanolamide, isostearic acid monoisopropanolamide (isostearamide MIPA), erucic acid diethanolamide, ricinoleic acid monoethanolamide, coconut fatty acid monoisopropanolamide (cocoamide MIPA), coconut acid monoethanolamide (Cocamide MEA), palm kernel fatty acid diethanolamide, coconut fatty acid diethanolamide, lauric diethanolamide, polyoxyethylene coconut fatty acid monoethanolamide, coconut fatty acid monoethanolamide, lauric monoethanolamide, lauric acid monoisopropanolamide (lauramide MIPA), myristic acid monoisopropanolamide (Myristamide MIPA), coconut fatty acid diisopropanolamide (cocamide DIPA), and mixtures thereof.

In some instances, the fatty acid alkanolamides preferably include cocamide MIPA, cocamide DEA, cocamide MEA, cocamide DIPA, and mixtures thereof. In particular, the fatty acid alkanolamide may be cocamide MIPA, which is commercially available under the tradename EMPILAN from Innospec Active Chemicals.

Fatty acid alkanolamides include those of the following structure:

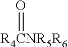

wherein R₄ is an alkyl chain of 4 to 20 carbon atoms (R₄ may be, for example, selected from lauric acid, coconut acid, palmitic acid, myristic acid, behenic acid, babassu fatty acid, isostearic acid, stearic acid, corn fatty acid, soy fatty acid, shea butter fatty acids, caprylic acid, capric acid, and mixtures thereof);
R₆ is selected from —CH₂OH, —CH₂CH₂OH, —CH₂CH₂CH₂OH, —CH₂(CHOH)₄CH₂OH, -benzyl, and mixtures thereof;
R₆ is selected from —H, —CH₃, —CH₂OH, —CH₂CH₃, —CH₂CH₂OH, —CH₂CH₂CH₂OH, —CH₂(CHOH)₄CH₂OH, -benzyl, and mixtures thereof.

In some instances, the one or more of the fatty acid alkanolamides include one or more acyl glucamides, for example, acyl glucamides having a carbon chain length of 8 to 20. Non-limiting examples include lauroyl/myristoyl methyl glucamide, capryloyl/capryl methyl glucamide, lauroyl methyl glucamide, myristoyl methyl glucamide, capryloyl methyl glucamide, capryl methyl glucamide, cocoyl methyl glucamide, capryloyl/caproyl methyl glucamide, cocoyl methyl glucamide, lauryl methylglucamide, oleoyl methylglucamide oleate, stearoyl methylglucamide stearate, sunfloweroyl methylglucamide, and tocopheryl succinate methylglucamide.

The compositions of the instant disclosure may include one or more alkyl polyglucosides. Non-limiting examples of alkyl polyglucosides include those having the following formula:

R¹—O—(R²O)ₙ−Z(x)
wherein R¹ is an alkyl group having 8-18 carbon atoms;
R² is an ethylene or propylene group;
Z is a saccharide group with 5 to 6 carbon atoms;
n is an integer from 0 to 10; and
x is an integer from 1 to 5.

Useful alkyl poly glucosides include lauryl glucoside, octyl glucoside, decyl glucoside, coco glucoside, caprylyl/capryl glucoside, and sodium lauryl glucose carboxylate. Typically, the at least one alkyl poly glucoside compound is selected from the group consisting of lauryl glucoside, decyl glucoside and coco glucoside. In some instances, decyl glucoside is particularly preferred.

The compositions of the instant disclosure may include one or more miscellaneous nonionic surfactants or emulsifiers. Nonlimiting examples include alcohols, alpha-diols, alkylphenols and esters of fatty acids, being ethoxylated, propoxylated or glycerolated and having at least one fatty chain comprising, for example, from 8 to 18 carbon atoms, it being possible for the number of ethylene oxide or propylene oxide groups to range from 2 to 50, and for the number of glycerol groups to range from 1 to 30. Maltose derivatives may also be mentioned. Non-limiting mention may also be made of copolymers of ethylene oxide and/or of propylene oxide; condensates of ethylene oxide and/or of propylene oxide with fatty alcohols; polyethoxylated fatty amides comprising, for example, from 2 to 30 mol of ethylene oxide; polyglycerolated fatty amides comprising, for example, from 1.5 to 5 glycerol groups, such as from 1.5 to 4; ethoxylated fatty acid esters of sorbitan comprising from 2 to 30 mol of ethylene oxide; ethoxylated oils from plant origin; fatty acid esters of sucrose; fatty acid esters of polyethylene glycol; polyethoxylated fatty acid mono or diesters of glycerol (C₆-C₂₄)alkylpolyglycosides; N—(C₆-C₂₄)alkylglucamine derivatives, amine oxides such as (C₁₀-C₁₄)alkylamine oxides or N—(C₁o-C₁₄)acylaminopropylmorpholine oxides; and mixtures thereof.

Such nonionic surfactants may preferably be chosen from polyoxyalkylenated or polyglycerolated nonionic surfactants. The oxyalkylene units are more particularly oxyethylene or oxypropylene units, or a combination thereof, and are preferably oxyethylene units.

In some cases, the nonionic surfactant may be selected from esters of polyols with fatty acids with a saturated or unsaturated chain containing for example from 8 to 24 carbon atoms, preferably 12 to 22 carbon atoms, and alkoxylated derivatives thereof, preferably with a number of alkyleneoxide of from 10 to 200, and more preferably from to 100, such as glyceryl esters of a C₈-C₂₄, preferably C₁₂-C₂₂, fatty acid or acids and alkoxylated derivatives thereof, preferably with a number of alkyleneoxide of from 10 to 200, and more preferably from 10 to 100; polyethylene glycol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sorbitol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sugar (sucrose, glucose, alkylglycose) esters of a C₈-C₂₄, preferably C₁₂-C₂₂, fatty acid or acids and alkoxylated derivatives thereof, preferably with a number of alkyleneoxide of from 10 to 200, and more preferably from to 100; ethers of fatty alcohols; ethers of sugar and a C₈-C₂₄, preferably C₁₂-C₂₂, fatty alcohol or alcohols; and mixtures thereof.

Examples of ethoxylated fatty esters that may be mentioned include the adducts of ethylene oxide with esters of lauric acid, palmitic acid, stearic acid or behenic acid, and mixtures thereof, especially those containing from 9 to 100 oxyethylene groups, such as PEG-9 to PEG-50 laurate (as the CTFA names: PEG-9 laurate to PEG-50 laurate); PEG-9 to PEG-50 palmitate (as the CTFA names: PEG-9 palmitate to PEG-50 palmitate); PEG-9 to PEG-50 stearate (as the CTFA names: PEG-9 stearate to PEG-50 stearate); PEG-9 to PEG-50 palmitostearate; PEG-9 to PEG-50 behenate (as the CTFA names: PEG-9 behenate to PEG-50 behenate); polyethylene glycol 100 EO monostearate (CTFA name: PEG-100 stearate); and mixtures thereof.

As glyceryl esters of fatty acids, glyceryl stearate (glyceryl mono-, di- and/or tristearate) (CTFA name: glyceryl stearate) or glyceryl ricinoleate and mixtures thereof can in particular be cited.

As glyceryl esters of C₈-C₂₄ alkoxylated fatty acids, polyethoxylated glyceryl stearate (glyceryl mono-, di- and/or tristearate) such as PEG-20 glyceryl stearate can for example be cited.

Mixtures of these surfactants, such as for example the product containing glyceryl stearate and PEG-100 stearate, marketed under the name ARLACEL 165 by Uniqema, and the product containing glyceryl stearate (glyceryl mono- and distearate) and potassium stearate marketed under the name TEG1N by Goldschmidt (CTFA name: glyceryl stearate SE), can also be used.

The total amount of the one or more nonionic surfactants or emulsifiers in the compositions, if present, will vary. Nonetheless, in various embodiments, the compositions include about 0.01 to about 10 wt. % of the one or more nonionic surfactants or emulsifiers. In further embodiments, the compositions include about 0.01 to about 8 wt. %, about 0.01 to about 5 wt. %, about 0.01 to about 3 wt. %, about 0.01 to about 1 wt. %, about 0.05 to about 10 wt. %, about 0.05 to about 8 wt. %, about 0.05 to about 5 wt. %, about 0.05 to about 3 wt. %, or about 0.05 to about 1 wt. %, about 0.1 to about 10 wt. %, about 0.1 to about 8 wt. %, about 0.1 to about 5 wt. %, or about 0.1 to about 3 wt. % based on a total weight of the composition.

### (h) Water

The total amount of water in the skin perfecting and tightening compositions will vary. Nonetheless, in various embodiments, the skin perfecting and tightening compositions include about 50 to about 90 wt. % of water, based on the total weight of the compositions. In further embodiments, the compositions include about 60 to about 90 wt. %, about 65 to about 90 wt. % about 70 to about 90 wt. %, about 75 to about 90 wt. %, about 80 to about 90 wt. %, about 50 to about 85 wt. %, about 60 to about 85 wt. %, about 65 to about 85 wt. %, about 70 to about 85 wt. %, about 75 to about 85 wt. %, based on a total weight of the composition.

### (i) Miscellaneous Ingredients

The skin perfecting and tightening compositions optionally include or exclude (or are essentially free from) one or more miscellaneous ingredients. Miscellaneous ingredients are ingredients that are compatible with the compositions and do not disrupt or materially affect the basic and novel properties of the compositions. Nonlimiting examples of ingredients include preservatives, fragrances, pH adjusters, salts, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, etc. In various embodiments, the miscellaneous ingredients are chosen from preservatives, fragrances, pH adjusters, salts, chelating agents, buffers, composition colorants, and mixtures thereof.

The total amount of the one or more miscellaneous ingredients in skin perfecting and tightening compositions, if present, will vary. Nonetheless, in various embodiments, the compositions include about 0.1 to about 15 wt. % of the one or more miscellaneous ingredients, based on the total weight of the compositions. In further embodiments, the compositions include about 0.1 to about 12 wt. %, about 0.1 to about 10 wt. %, about 0.1 to about 5 wt. %, about 0.5 to about 15 wt. %, about 0.5 to about 12 wt. %, about 0.5 to about 10 wt. %, about 0.5 to about 8 wt. %, about 0.5 to about 5 wt. %, about 1 to about 15 wt. %, about 1 to about 12 wt. %, about 1 to about 10 wt. %, about 1 to about 8 wt. %, about 1 to about 5 wt. %, about 2 to about 15 wt. %, about 2 to about 12 wt. %, about 2 to about 10 wt. %, about 2 to about 8 wt. %, or about 2 to about 5 wt. %, based on the total weight of the compositions.

As already noted, antioxidants, skin active agents, depigmenting agents, anti-wrinkle agents, or mixtures thereof may be included as one or more of the miscellaneous ingredients. The antioxidants, skin active agents, depigmenting agents, anti-wrinkle agents, or mixtures thereof in the skin perfecting and tightening compositions, if present, may be in an amount from greater than zero to about 9 wt. %, greater than zero to about 8 wt. %, greater than zero to about 7 wt. %, greater than zero to about 6 wt. %, greater than zero to about 5 wt. %, greater than zero to about 4 wt. %, greater than zero to about 3 wt. %, greater than zero to about 2 wt. %; about 10 ppm to about 10 wt. % (100,000 ppm), about 10 ppm to about 5 wt. % (50,000 ppm), about 10 ppm to about 2.5 wt. % (25,000 ppm), about 10 ppm to about 1 wt. % (10,000 ppm), about 10 ppm to about 0.5 wt. % (5,000 ppm), about 10 ppm to about 0.3 wt. % (3,000 ppm), about 10 ppm to about 0.2 wt. % (2,000 ppm), about 10 ppm to about 0.1 wt. % (1,000 ppm), about 10 ppm to 500 ppm; about 0.1 to about 10 wt. %, about 0.1 to about 5 wt. %, about 0.1 to about 2.5 wt. %, about 0.1 to about 1 wt. %, about 0.1 to about 0.5 wt. %; about 1 to about 10 wt. %, about 1 to about 8 wt. %, about 1 to about 6 wt. %, about 1 to about 5 wt. %, about 1 to about 4 wt. %, about 1 to about 3 wt. %; about 2 to about 10 wt. %, about 2 to about 8 wt. %, about 2 to about 6 wt. %, about 2 to about 5 wt. %, about 2 to about 4 wt. %; about 3 to about 10 wt. %, about 3 to about 8 wt. %, about 3 to about 6 wt. %, about 3 to about 5 wt. %; about 4 to about 10 wt. %, about 4 to about 8 wt. %, or about 4 to about 6 wt. %, based on the total weight of the skin perfecting and tightening composition.

pH

The pH of the skin perfecting and tightening composition can vary but is typically less than 7, i.e., it is acidic. In various embodiments, the pH of the skin perfecting and tightening compositions is from about 3 to less than 7, about 3.5 to less than 7, about 4 to less than 7, about 3 to about 6.5, about 3.5 to about 6.5, about 4 to about 6.5, about 3 to about 6, about 3.5 to about 6, about 4 to about 6, about 3 to about 5.5, or about 3.5 to about 5.5.

### Methods

The instant disclosure also relates to methods of treating skin. The methods include applying the skin perfecting and tightening compositions to the skin. The skin perfecting and tightening compositions are typically applied directly to the skin using the hands or a cloth. The skin may be optionally washed or rinsed prior to application. The method for treating the skin can be carried out once daily or may be carried out multiple times per day. For example, the method for treating skin may be carried out once daily, twice daily, weekly, bi-weekly; and may be carried out for an extended period of time, for example, for 1, 2, 3, 4, 5, or 6 months. The methods dissolve dead skin cells, improve the appearance of fine lines and discoloration, remove excess oil from the skin, improve skin texture and tone, and/or hydrates the skin.

### Natural or Naturally Derived

In various embodiments, the skin smoothing and tightening compositions comprise, consist, or substantially consist of naturally derived ingredients. For purposes of the instant disclosure the term “naturally derived ingredient” is an ingredient that is not derived from petrochemical or fossil fuel sources. The naturally derived ingredients (bio-ingredients) are formed from “natural” feedstock. Such feedstocks are referred to herein as “natural” and “renewable” (i.e., “sustainable”) and are known as a non-petroleum-derived feedstock. Further, such materials are formed by “new” carbon and not from petroleum or other fossil fuel sources (“old” carbon). Such products are referred to herein as “natural” products and are known in the art as non-petrochemically-derived or “bio” products. Natural and naturally derived ingredients or compounds are sustainable because they come from renewable sources, and not those that deplete a limited natural resource, such as a fossil fuel or other non-renewable resource such as petroleum. Thus, a natural or naturally derived compound is not petrochemically derived, and/or is made from sources that are not petrochemically derived would be sustainable and renewable.

Naturally derived ingredients (bio-compounds or bio-ingredients) are formed using biomass (e.g., material stored from carbon cycle processes in living plants and roots, etc. or released through animal respiration or through decomposition). When carbon decomposes and is broken down over millions of years under pressure, it creates fossil fuels (the source of petrochemically-derived carbon). Bio-compounds or bio-ingredients used herein are intended to include materials derived from the carbon of plant sources/biomass, that exist recently and are also sustainable, and are not derived from fossil fuels.

The bio-based or “natural” products from such feedstocks may be tested to determine that they come from a true, natural and sustainable (as those terms are defined herein) feedstock source. Some products are known or advertised as being from natural sources when, in fact, they may not be prepared from truly natural and/or sustainable feedstock. A natural organic product is typically defined as a compound produced naturally by a living organism. To distinguish a petroleum-based product from a truly natural and/or sustainable product, one must test for the authenticity using established and credible test methods. The most current method employs a detailed analysis of stable isotopes using mass spectroscopy and evaluating carbon-12/carbon-13 and/or hydrogen-1/hydrogen-2 ratios. Such testing is available through several analytical service testing organizations and is much faster, more cost effective, and yields more detailed information compared to radiocarbon testing methods.

Stable isotope analysis is based on the principle of kinetic isotope effect. The latter effect is well-known to those in the art of chemical kinetics. In the broadest terms, heavy isotopes of a particular element react slower than their lighter equivalent (e.g., carbon-12 as opposed to carbon-13). So, as plants incorporate carbon dioxide into their biomass, the ratio of carbon-12 to carbon-13 will vary depending on the type of chemistry used in the plant to make biomass (e.g., whether the plant undergoes a C₃ or C₄ photosynthesis pathway). This is commonly reported as the δ¹³C/¹²C ratio (i.e., δ¹³C), and is referenced to a current carbon dioxide standard. In addition, similar isotope kinetic effects are observed when water is incorporated into new biomass, and this is measured as the δ²H/¹H ratio (i.e., δ²H). Using a combination of δ¹³C and δ²H ratios, one familiar with in the relevant art is able to readily distinguish and validate the nature of the feedstock that was used to prepare the product being analyzed (i.e., whether it is petrochemically-derived or derived from recently living or living algae-, plant- or similar bio-sources).

Because petroleum-based (i.e., petrochemically-derived) feedstocks are derived from plants and animals buried millions of years ago, the feedstocks' radiocarbon (i.e., ¹⁴C) has been lost to decay. The ASTM International standards provide testing standards to determine the authenticity of a “bio-based compound” using radiocarbon, which may be found in ASTM D6866-16. This standard distinguishes newer carbon from carbon derived from fossil-fuel, or petroleum- and petrochemically-derived sources, i.e., “old carbon.” The amount of ¹⁴C in recent or current biomass is known, so a percentage of carbon from a renewable source can be estimated from a total organic carbon analysis, which provides the data necessary to determine if a compound is truly derived from a “natural” and/or “sustainable” (“renewable”) feedstock source or is derived conversely from a compound of “old” sequestration (i.e., a petrochemically-derived or petroleum-based source). The use of petroleum-based or often labeled fossil-based feedstocks is generally accepted as being non-sustainable, i.e., old carbon is a non-sustainable and not a renewable feedstock, and furthermore, is not considered “natural” and “sustainable” in the art. As defined herein, it would not be considered to be a “natural” product or useful in a “natural” formulation. Hence, use of such feedstocks does not represent a path forward to development of “natural” and “renewable” alternative preservatives.

In various embodiments, the skin smoothing and tightening composition are “natural” compositions. For purposes of the instant disclosure, a composition is a “natural” composition provided that at least 90 wt. % of the composition consists of “naturally derived ingredients,” relative to a total weight of the composition, i.e., at least 90 wt. % of the composition consists of ingredient that are not petrochemically derived. In further embodiments, the natural composition consists of at least 95 wt. %, 96 wt. %, 97 wt. %, 98 wt. %, 99 wt. %, or 100 wt. % of naturally derived ingredients, relative to a total weight of the composition.

### Exclusions

All components that are positively set forth in the instant disclosure may be negatively excluded from the claims, e.g., a claimed composition may be “free,” “essentially free” (or “substantially free”) of one or more components that are positively set forth in the instant disclosure.

In various embodiments, the skin smoothing and tightening composition is free or essentially free from anionic surfactants, cationic surfactants, or both anionic and cationic surfactants.

In various embodiments, the skin smoothing and tightening composition is free or essentially free from polymers, copolymers, and crosspolymers formed with acrylate or methacrylate monomers, e.g, free or essentially free from polyacrylic acid and polyacrylate polymers and crosspolymers.

In various embodiments, the skin smoothing and tightening composition is free or essentially free from polyorganosiloxanes (silicones), for example, dimethicone. In various embodiments, the skin smoothing and tightening composition is free or essentially free from amino silicones (or amino-functionalized silicones), for example, amodimethicone.

In further embodiments, the skin smoothing and tightening composition is free or essentially free from monosaccharides and disaccharides. For example, the composition is free or essentially free from ribose, arabinose, glucose, fructose, xylose, sucrose, and/or methyl glucoside.

In various embodiments, the skin smoothing and tightening compositions are free or essentially free from synthetic ingredients. For the purposes of the instant disclosure, the term “synthetic ingredient” means a compound that is neither naturally existing nor a derivative of a natural compound. Such compound must not necessarily be obtained naturally or derived from a natural compound provided the compound occurs in nature or can be derived from a compound that occurs in nature. In various embodiments, the skin perfecting and tightening compositions comprise less than 25 wt. % of synthetic ingredients. In further embodiments, the skin perfecting and tightening composition comprise less than 20 wt. %, less than 15 wt. %, less than 10 wt. %, less than 5 wt. %, less than 4 wt. %, less than 3 wt. %, less than 2 wt. %, or less than 1 wt. % of synthetic ingredients.

In various embodiments, the skin smoothing and tightening composition is free or essentially free from synthetic polymers, for example, synthetic cationic polymers, synthetic thickening polymers, synthetic film forming polymers, or a combination thereof.

In various embodiments, the skin smoothing and tightening composition is free or essentially free from fatty alcohols. In various embodiments, the skin smoothing and tightening composition is free or essentially free from fatty acids. In various embodiments, the skin smoothing and tightening composition is free or essentially free from fibers or filaments. In various embodiments, the skin smoothing and tightening composition is free or essentially from proteins, peptides, amino acids (for example, arginine, histidine, methionine, lysine, proline, leucine, and glycine, etc.), or a combination thereof.

### EMBODIMENTS

In various embodiments, the skin smoothing and tightening compositions comprise or consist of:
(a) about 1 to about 12 wt. %, preferably about 2 to about 10, more preferably about 3 to about 8 wt. % of one or more polysaccharides with amine groups, preferably chitosan;
(b) about 0.1 to about 8 wt. %, preferably about 0.5 to about 6 wt. %, more preferably about 1 to about 5 wt. % of silica particles,
   wherein (a) and (b) are in a weight ratio of about 6:1 to about 1:1, preferably about 5:1 to about 1.5:1, more preferably about 4:1 to about 2:1.
(c) about 0.1 to about 6 wt. %, preferably about 0.5 to about 5 wt. %, more preferably about 1 to about 4 wt. % of one or more mineral acids, organic acids, salts thereof, or a combination thereof, preferably one or more alpha hydroxy acids, salts thereof, or a combination thereof, preferably wherein the one or more alpha hydroxy acids are selected from glycolic acid, lactic acid, tartaric acid, mandelic acid, malic acid, citric acid, phytic acid, hydroxycaprylic acid, hydroxycapric acid, a salt thereof, or a combination thereof, more preferably wherein the one or more alpha hydroxy acid comprises or consists of glycolic acid, a salt thereof, or a combination thereof;
(d) optionally, about 1 to about 40 wt. %, preferably about 1 to about 20 wt. %, more preferably about 1 to about 10 wt. % of one or more fatty compounds;
(e) optionally, about 0.01 to about 25 wt. %, preferably about 0.1 to about 15 wt. %, more preferably about 0.5 to about 10 wt. % of one or more water-soluble solvents, preferably wherein the one or more water soluble solvents are selected from glycerin, C₁-C₆ mono-alcohols, polyols (polyhydric alcohols), glycols, or a mixture thereof, even more preferably wherein the one or more water soluble solvents comprises or consists of glycerin;
(f) optionally, about 0.1 to about 6 wt. %, preferably about 0.5 to about 5 wt. %, more preferably about 1 to about 4 wt. % of one or more thickening agents;
(g) optionally, about 0.01 to about 10 wt. %, preferably about 0.1 to about 8 wt. %, more preferably of one or more nonionic surfactants or emulsifiers, preferably wherein the one or more nonionic surfactants or emulsifiers are selected from nonionic surfactants or emulsifiers are selected from alkoxylated fatty alcohols, fatty acid esters of polyoxyethylene glycol, ethoxylated mono or diglycerides, sorbitan esters, ethoxylated sorbitan esters, fatty acid glycol esters, ethylene oxide, alkyl(ether)phosphates, alkylpolyglucosides, and mixtures thereof, preferably wherein the one or more nonionic surfactants or emulsifiers include one or more polyoxyethylenated alkyl and polyalkyl esters of sorbitan (e.g., polysorbates); and
(h) about 60 to about 90 wt. %, preferably about 70 to about 90 wt. %, more preferably about 80 to about 90 wt % of water;
(i) optionally, about 0.01 to about 10, preferably about 0.1 to about 8, more preferably about 1 to about 5 wt. % of one or more miscellaneous ingredients, preferably wherein the one or more miscellaneous ingredient are selected from preservatives, fragrances, pH adjusters, salts, chelating agents, buffers, antioxidants, flavonoids, vitamins, amino acids, botanical extracts, UV filtering agents, peptides, proteins, protein hydrolysates, and/or isolates, fillers (e.g., organic and/or inorganic fillers such as talc, calcium carbonate, or other particular materials, etc.), emollients, composition colorants, depigmenting agents, skin active agents, anti-wrinkle agents or a mixture thereof;
   wherein all weight percentages are based on a total weight of the composition; and wherein the composition is preferably an oil in water emulsion.

In a further embodiment, the skin perfecting and tightening composition comprises or consists of:
(a) about 1 to about 12 wt. %, preferably about 2 to about 10, more preferably about 3 to about 8 wt. % of chitosan;
(b) about 0.1 to about 8 wt. %, preferably about 0.5 to about 6 wt. %, more preferably about 1 to about 5 wt. % of silica particles,
   wherein (a) and (b) are in a weight ratio of about 6:1 to about 1:1, preferably about 5:1 to about 1.5:1, more preferably about 4:1 to about 2:1;
(c) about 0.1 to about 6 wt. %, preferably about 0.5 to about 5 wt. %, more preferably about 1 to about 4 wt. % of one or more alpha hydroxy acids, salts thereof, or a combination thereof, preferably wherein the one or more alpha hydroxy acids are selected from glycolic acid, lactic acid, tartaric acid, mandelic acid, malic acid, citric acid, phytic acid, hydroxycaprylic acid, hydroxycapric acid, a salt thereof, or a combination thereof, more preferably wherein the one or more alpha hydroxy acid comprises or consists of glycolic acid, a salt thereof, or a combination thereof;
(d) about 1 to about 40 wt. %, preferably about 1 to about 20 wt. %, more preferably about 1 to about 10 wt. % of one or more fatty compounds;
(e) about 0.01 to about 25 wt. %, preferably about 0.1 to about 15 wt. %, more preferably about 0.5 to about 10 wt. % of one or more water-soluble solvents, preferably wherein the one or more water soluble solvents are selected from glycerin, C₁-C₆ mono-alcohols, polyols (polyhydric alcohols), glycols, or a mixture thereof, even more preferably wherein the one or more water soluble solvents comprises or consists of glycerin;
(f) about 0.1 to about 6 wt. %, preferably about 0.5 to about 5 wt. %, more preferably about 1 to about 4 wt. % of hydroxyethylcellulose, hydroxypropylcellulose, hydroxyethylmethylcellulose, hydroxypropylmethylcellulose, carboxymethyl cellulose, derivatives thereof, or a combination thereof;
(g) about 0.01 to about 10 wt. %, preferably about 0.1 to about 8 wt. %, more preferably of one or more nonionic surfactants or emulsifiers, preferably wherein the one or more nonionic surfactants or emulsifiers are selected from nonionic surfactants or emulsifiers are selected from alkoxylated fatty alcohols, fatty acid esters of polyoxyethylene glycol, ethoxylated mono or diglycerides, sorbitan esters, ethoxylated sorbitan esters, fatty acid glycol esters, ethylene oxide, alkyl(ether)phosphates, alkylpolyglucosides, and mixtures thereof, preferably wherein the one or more nonionic surfactants or emulsifiers include one or more polyoxyethylenated alkyl and polyalkyl esters of sorbitan (e.g., polysorbates); and
(h) about 60 to about 90 wt. %, preferably about 70 to about 90 wt. %, more preferably about 80 to about 90 wt % of water;
(i) optionally, about 0.01 to about 10, preferably about 0.1 to about 8, more preferably about 1 to about 5 wt. % of one or more miscellaneous ingredients, preferably wherein the one or more miscellaneous ingredient are selected from preservatives, fragrances, pH adjusters, salts, chelating agents, buffers, antioxidants, flavonoids, vitamins, amino acids, botanical extracts, UV filtering agents, peptides, proteins, protein hydrolysates, and/or isolates, fillers (e.g., organic and/or inorganic fillers such as talc, calcium carbonate, or other particular materials, etc.), emollients, composition colorants, depigmenting agents, skin active agents, anti-wrinkle agents or a mixture thereof;
   wherein all weight percentages are based on a total weight of the composition; and wherein the composition is preferably an oil in water emulsion.

Implementation of the present disclosure is provided by way of the following examples. The examples serve to illustrate the technology without being limiting in nature.

EXAMPLE 1

| | | | A |
|---|---|---|---|
| | | | wt. % |
| (a) | Polysaccharide | CHITOSAN | 4.5 |
| | with an amine | | |
| | group | | |
| (b) | Silica | SILICA | 2 |
| Ratio of (a):(b) | | | 2.3 |
| (c) | Alpha-Hydroxy | GLYCOLIC ACID | 1.4 |
| | Acid | | |
| (d) | Fatty | | 4 |
| | Compound | | |
| (e) | Water-Soluble | GLYCERIN | 1 |
| | Solvent | | |
| (f) | Thickening | | 1.3 |
| | Agent | | |
| (g) | Nonionic | POLYSORBATE 20 | 0.4 |
| | Surfactant or | | |
| | Emulsifier | | |
| (i) | pH Adjuster | CITRIC ACID | — |
| Misc. | Preservative | PHENOXYETHANOL | 0.5 |
| (h) | Water | WATER | 85 |

The foregoing description illustrates and describes the disclosure. Additionally, the disclosure shows and describes only the preferred embodiments. However, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

As used herein, the terms “comprising,” “having,” and “including” are used in their open, non-limiting sense.

The terms “a,” “an,” and “the” are understood to encompass the plural as well as the singular. Thus, the term “a mixture thereof” also relates to “mixtures thereof.” Throughout the disclosure, the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.” Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting. Appropriate counterions for the components described herein are known in the art.

The expression “one or more” means “at least one” and thus includes individual components as well as mixtures/combinations.

The term “plurality” means “more than one” or “two or more.”

The term “chitosan” means any copolymer formed of constituent units N-acetyl-D-glucosamine and D-glucosamine, whose degree of acetylation is less than 90%, preferably less than 80%, preferably less than 70%, preferably less than 60%, preferably less than 50%. In further embodiments, the degree of acetylation is sufficient to ensure solubility of at least 70%, preferably at least 80%, preferably at least 90%, preferably at least 95%, preferably at least 99% of the chitosan in the skin tightening and perfecting composition at 25° C.

Other than in the operating examples, or where otherwise indicated, all numbers expressing quantities of ingredients and/or reaction conditions may be modified in all instances by the term “about,” meaning within +/−5% of the indicated number.

All percentages, parts and ratios herein are based upon the total weight of the compositions of the present invention, unless otherwise indicated.

Some of the various categories of components identified may overlap. In such cases where overlap may exist and the composition includes both components (or the composition includes more than two components that overlap), an overlapping compound does not represent more than one component. For example, certain compounds may be considered both a nonionic surfactant or emulsifier and a fatty compound. If a particular composition includes both a nonionic surfactant or emulsifier and a fatty compound, a single compound will serve as only the nonionic surfactant or emulsifier or only as the fatty compound (the single compound does not simultaneously serve as both the nonionic surfactant or emulsifier and the fatty component).

A “rinse-off” product refers to a composition that is rinsed and/or washed from the skin with water either after or during the application of the composition onto the skin. At least a portion of the composition is removed from the skin during the rinsing and/or washing.

A “leave-on” product refers to a composition that is not rinsed and/or washed from the skin after or during application of the composition onto the skin. The composition remains on the skin throughout the day and/or night.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

The composition of the instant case optionally includes one or more surfactants and/or emulsifiers, for example, one or more nonionic, anionic, cationic, and/or amphoteric/zwitterionic surfactants. The term “surfactants” and “emulsifiers” include salts of the surfactants and emulsifiers even if not explicitly stated. In other words, whenever the disclosure refers to a surfactant or emulsifier, it is intended that salts are also encompassed to the extent such salts exist, even though the specification may not specifically refer to a salt (or may not refer to a salt in every instance throughout the disclosure), for example, by using language such as “a salt thereof” or “salts thereof.” Sodium and potassium are common cations that form salts with surfactants and emulsifiers. However, additional cations such as ammonium ions, or alkanolammonium ions such as monoethanolammonium or triethanolammonium ions, may also form salts of surfactants.

The term “substantially free” or “essentially free” as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, or none of the specified material.

All components that are positively set forth in the instant disclosure may be negatively excluded from the claims, e.g., a claimed composition may be “free,” “essentially free” (or “substantially free”) of one or more components that are positively set forth in the instant disclosure.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A skin perfecting composition comprising:
(a) about 1 to about 12 wt. % of one or more polysaccharides with amine groups;
(b) about 0.1 to about 8 wt. % of silica particles,
wherein (a) and (b) are in a weight ratio of about 6:1 to about 1:1;
(c) about 0.1 to about 6 wt. % of one or more acids, salts thereof, or a combination thereof;
(d) optionally, about 1 to about 40 wt. % of one or more fatty compounds; and
(e) optionally, about 0.01 to about 25 wt. % of one or more water-soluble solvents;
(h) about 50 to about 90 wt. % of water;
wherein all weight percentages are based on a total weight of the composition.

2. The composition of claim 1, wherein the one or more polysaccharide with amine groups are selected from polyhexosamines of formula (B), organic or mineral acid salts thereof, a or p anomers thereof, isomers thereof of L or D configuration, and solvates/hydrates thereof: 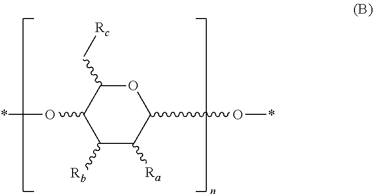
Rₐ, R_{b}, R_{c} of each saccharide unit may be identical or different;
n is an integer greater than or equal to 2;
Rₐ, R_{b}, and R_{c}, which are identical or different, represent:
i) a hydroxyl group,
ii) a (C₁-C₄)alkoxy group, the alkyl group of which may be optionally substituted with one or more hydroxyl groups,
iii) a carboxyl group, and
iv) an NR₁R₂ group, wherein R₁ and R₂ represent:
i) a hydrogen atom or ii) —C(O)—R′₁ with R′₁ representing a (C₁-C₄)alkyl group;
wherein at least one of the Rₐ, R_{b} or R_{c} represents an NR₁R₂ group and that at least one of the NR₁R₂ groups represents an NH₂ group.

3. The composition of claim 1, wherein the one or more polysaccharide with amine groups are selected from polyhexosamines of formula (B₁), organic or mineral acid salts thereof, a or p anomers thereof, isomers thereof of L or D configuration, and solvates/hydrates thereof: 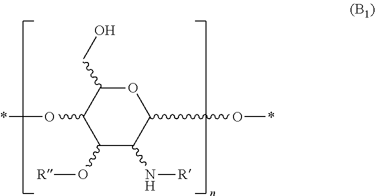
R′ represents a hydrogen atom or a (C₁-C₄)alkylcarbonyl group;
R″ represents a hydrogen atom or a (C₁-C₄)alkyl group optionally substituted with a carboxyl group; and
n is an integer greater than or equal to 2.

4. The composition of claim 1, wherein the one or more polysaccharides with amine groups are selected from polyhexosamines of formula (B₃), organic or mineral acid salts thereof, and solvates/hydrates thereof:

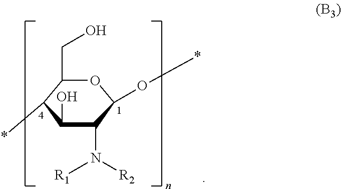

5. The composition of claim 1, wherein the one or more polysaccharide with amine groups are selected from polyhexosamines of formula (B₄), organic or mineral acid salts thereof, and solvates/hydrates thereof: 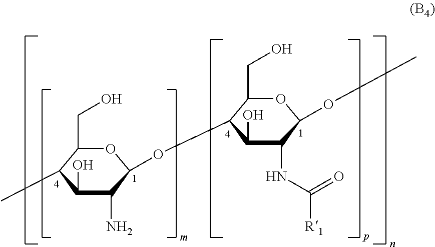
R′₁ representing a (C₁-C₄)alkyl group such as methyl;
p is greater than 0 and ranges up to 0.5; and
n is an integer greater than or equal to 2.

6. The composition of claim 1, wherein the one or more polysaccharides with amine groups comprises chitosan or is chitosan.

7. The skin perfecting composition of claim 6, wherein the one or more silica particles are hydrophilic silica particles.

8. The composition of claim 7, wherein the one or more hydrophilic silica particles are selected from fumed silica, precipitated silica, or a combination thereof.

9. The composition of claim 1, further comprising:
(f) about 0.1 to about 6 wt. % of one or more thickening agents; and/or
(g) about 0.01 to about 10 wt. % of one or more nonionic surfactants or emulsifiers.

10. The composition of claim 1, wherein the one or more acids include or consist of alpha hydroxy acids.

11. The composition of claim 1, wherein the one or more fatty compounds are in an amount of about 1 to about 10 wt. %.

12. The composition of claim 1 comprising one or more water soluble solvents selected from glycerin, C₁-C₆ mono-alcohols, polyols (polyhydric alcohols), glycols, or a mixture thereof.

13. The composition of claim 9 comprising the one or more thickening agents.

14. The composition of claim 1, wherein the one or more thickening agents is in an amount of 0.5 to about 3 wt. %.

15. The composition of claim 1, wherein the one or more nonionic surfactants or emulsifiers are selected from alkoxylated fatty alcohols, fatty acid esters of polyoxyethylene glycol, ethoxylated mono or diglycerides, sorbitan esters, ethoxylated sorbitan esters, fatty acid glycol esters, ethylene oxide, alkyl(ether)phosphates, alkylpolyglucosides, and mixtures thereof.

16. The composition of claim 1, further comprising:
(i) about 0.01 to about 10 wt. % of one or more miscellaneous ingredients.

17. The composition of claim 1 having a pH of about 3 to about 6.

18. The composition of claim 1 in the form of an oil in water emulsion.

19. A skin perfecting composition comprising:
(a) about 1 to about 12 wt. % of chitosan;
(b) about 0.1 to about 10 wt. % of hydrophilic fumed or precipitated silica nano or microparticles;
wherein (a) and (b) are in a weight ratio of about 10:1 to about 1:1;
(c) about 0.1 to about 5 wt. % of glycolic acid, lactic acid, tartaric acid, mandelic acid, malic acid, citric acid, phytic acid, hydroxycaprylic acid, hydroxycapric acid, a salt thereof, or a combination thereof;
(d) about 1 to about 20 wt. % of one or more fatty compounds;
(e) about 0.1 to about 25 wt. % of one or more water-soluble solvents selected from glycerin, C₁-C₆ mono-alcohols, polyols (polyhydric alcohols), glycols, or a mixture thereof;
(f) about 0.1 to about 5 wt. % of one or more thickening agents;
(g) about 0.01 to about 5 wt. % of one or more nonionic surfactants or emulsifiers selected from alkoxylated fatty alcohols, fatty acid esters of polyoxyethylene glycol, ethoxylated mono or diglycerides, sorbitan esters, ethoxylated sorbitan esters, fatty acid glycol esters, ethylene oxide, alkyl(ether)phosphates, alkylpolyglucosides, and mixtures thereof;
(h) about 50 to about 90 wt. % of water; and
(i) optionally, about 0.01 to about 10 wt. % of one or more miscellaneous ingredients selected from preservatives, fragrances, pH adjusters, salts, chelating agents, buffers, antioxidants, flavonoids, vitamins, amino acids, botanical extracts, UV filtering agents, peptides, proteins, protein hydrolysates, and/or isolates, fillers (e.g., organic and/or inorganic fillers such as talc, calcium carbonate, or other particular materials, etc.), emollients, composition colorants, depigmenting agents, skin active agents, anti-wrinkle agents or a mixture thereof;
wherein all weight percentages are based on a total weight of the composition.

20. A method for improving the appearance of skin comprising applying the skin perfecting composition of claim 1 to the skin.
